(19) United States
(12) Patent Application Publication
(10) Pub. No.: US 2026/0092886 A1
(43) Pub. Date: Apr. 2, 2026

(54) GAS DETECTION CHIP AND MANUFACTURING METHOD THEREOF

(71) Applicant: National Taiwan Normal University, TAIPEI CITY (TW)

(72) Inventors: TIEN-LI CHANG, TAIPEI CITY (TW); SONG-PU HUANG, TAIPEI CITY (TW); QI-XUAN WU, TAIPEI CITY (TW); YUAN-JUN CHEN, TAIPEI CITY (TW)

(21) Appl. No.: 18/987,182

(22) Filed: Dec. 19, 2024

(30) Foreign Application Priority Data
Sep. 30, 2024 (TW) 113137229

(51) Int. Cl. G01N 27/12; G01N 33/00

(52) U.S. Cl. CPC G01N 27/127; G01N 33/0027

(57) ABSTRACT

A technology using laser thin-film electrode structure processes in a gas detection chip is provided. Different from traditional chemical etching or long pulse laser processing methods, a manufacturing method of the gas detection chip uses an ultrafast laser process to manufacture thin-film composite structures in gas detection development, and employs a multi-scale composite structure made of flexible materials (e.g., polyimide substrates) for gas detection. The gas detection chip prepared by the manufacturing method meets the demand for portable micro gas detection.

### CROSS-REFERENCE TO RELATED PATENT APPLICATION

This application claims the benefit of priority to Taiwan Patent Application No. 113137229, filed on Sep. 30, 2024. The entire content of the above identified application is incorporated herein by reference.

Some references, which may include patents, patent applications and various publications, may be cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference was individually incorporated by reference.

### FIELD OF THE DISCLOSURE

The present disclosure relates to a detection chip, and more particularly to a gas detection chip and a manufacturing method thereof.

### BACKGROUND OF THE DISCLOSURE

In recent years, gas detection technology has been developed at a considerable rate. In addition to improving on the relevant art, the range of applications of the gas detection devices has also been expanded. The gas detection devices is manufactured by adsorbing the gas to be measured on the surface, catalyzing the gas to produce a change in resistance, using an amplifier to generate a signal from this change, and finally analyzing the signal to determine a concentration of the detected gas. Studies to detect nitric oxide (NO) concentrations in the human body have been conducted in Europe, the United States, and other countries, finding that when a human is sick, the concentration of specific gases will increase. Accordingly, if gas sensors are made portable and integrated into mobile devices, they can be designed to effectively diagnose human health and achieve real-time monitoring.

As various types of functional detection chips have become increasingly difficult to develop in terms of flexibility and portability, materials that offer more flexibility have attracted widespread attention. However, due to physical characteristics of the materials, they are prone to damaging a substrate during traditional manufacturing processes (i.e., chemical etching, grinding, etc.), which in turn reduces the stability of the substrate. Compared to the traditional manufacturing processes, a non-contact process involving the use of an ultrafast laser process to manufacture multi-scale thin-film gas detection chips based on flexible substrates is being proposed, which does not require complex steps involved in the chemical etching. This approach can improve dimensional accuracy, increase process efficiency, and eliminate the use of hazardous chemical solvents, thereby enhancing the safety of manufacturing personnel and offering new prospects to the development of current detection chips.

### SUMMARY OF THE DISCLOSURE

In response to the above-referenced technical inadequacies, the present disclosure provides a technology using laser thin-film electrode structure processes in a gas detection chip. Different from traditional chemical etching or long pulse laser processing methods, a manufacturing method of the gas detection chip uses an ultrafast laser process to manufacture thin-film composite structures in gas detection development, and employs a multi-scale composite structure made of flexible materials (e.g., polyimide substrates) for gas detection. The gas detection chip prepared by the manufacturing method meets the demand for portable micro gas detection, and can be applied in detection applications for fields such as clinical medicine, electric vehicles, new energy, aerospace, and next-generation 5G communications technology.

In order to solve the above-mentioned problems, one of the technical aspects adopted by the present disclosure is to provide a method for producing a gas detection chip. The method for producing the gas detection chip includes: providing a substrate; forming a gas sensing material on the substrate; and using an ultrafast laser to cut and remove the gas sensing material from the substrate along a predetermined path to form an electrode pattern.

In order to solve the above-mentioned problems, another one of the technical aspects adopted by the present disclosure is to provide a gas detection chip. The gas detection chip includes a substrate, and an electrode pattern formed on the substrate. The electrode pattern has a micro-nano structure, and the electrode pattern is formed of a gas sensing material cut by pulses of an ultrafast laser.

Therefore, in the gas detection chip and manufacturing method thereof provided by the present disclosure, by virtue of the above technical solution, the gas detection chip prepared through the above method can be applied to NO gas detection and exhibits high sensitivity.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The described embodiments may be better understood by reference to the following description and the accompanying drawings, in which:

FIG. 1 is a first flowchart of a method for producing a gas detection chip according to an embodiment of the present disclosure;

FIG. 2 is a second flowchart of the method for producing the gas detection chip according to the embodiment of the present disclosure;

FIG. 3 is a third flowchart of the method for producing the gas detection chip according to the embodiment of the present disclosure;

FIG. 4 is a fourth flowchart of the method for producing the gas detection chip according to the embodiment of the present disclosure;

FIG. 5A is a schematic diagram of an electrode pattern according to first embodiments of the present disclosure;

FIG. 5B is a schematic diagram of the electrode pattern according to second embodiments of the present disclosure;

FIG. 5C is a schematic diagram of the electrode pattern according to third embodiments of the present disclosure;

FIG. 6A is a diagram illustrating the electrode pattern according to the first embodiment of FIG. 5A;

FIG. 6B is a partially enlarged diagram of the electrode pattern of the first embodiment;

FIG. 6C shows distribution state of the nanowires on the graphene layer according to the embodiment of the present disclosure;

FIG. 7A, FIG. 7B, and FIG. 7C are heating response curves of the electrode pattern under different voltages according to different embodiments of the present disclosure; and

FIG. 8A, FIG. 8B, FIG. 8C and FIG. 8D are detection sensitivity data of the gas detection chips prepared by the electrode patterns of different embodiments when detecting nitrogen oxides according to different embodiments of the present disclosure.

### DETAILED DESCRIPTION OF THE EXEMPLARY EMBODIMENTS

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a,” “an” and “the” includes plural reference, and the meaning of “in” includes “in” and “on.” Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first,” “second” or “third” can be used to describe various components, signals or the like, which are for distinguishing one component/signal from another one only, and are not intended to, nor should be construed to impose any substantive limitations on the components, signals or the like.

### [Method for Producing a Gas Detection Chip]

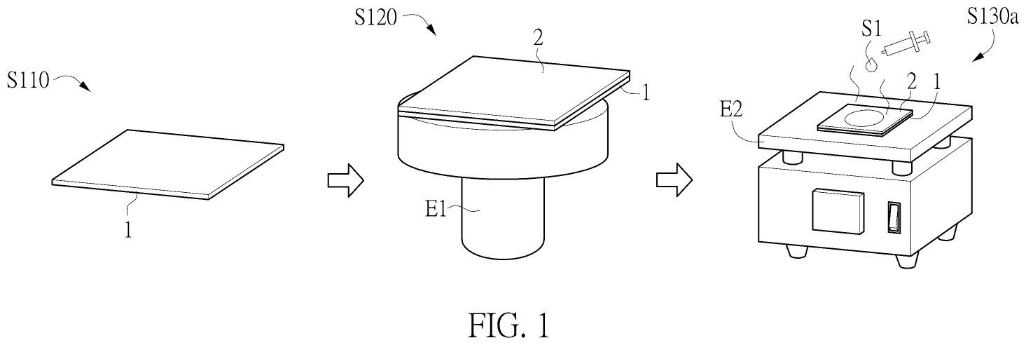

As shown in FIG. 1 to FIG. 4, an embodiment of the present disclosure provides a method for producing a gas detection chip, and more particularly to the method for producing the gas detection chip based on an ultrafast laser. The method for producing the gas detection chip includes: providing a substrate 1; forming a gas sensing material on the substrate 1; and using the ultrafast laser L to cut and remove the gas sensing material from the substrate 1 along a predetermined path to form an electrode pattern E. The gas sensing material is selected from the group consisting of graphene and metal oxide (e.g., zinc oxide, nickel oxide, tin dioxide and other metal oxides), and the gas sensing material has surface topography that includes at least one of: nanowires, nanoparticles, and nanosheets. The electrode pattern E includes at least one of a serpentine shape, a square shape, and a circular shape. In the present embodiment, the gas sensing material is formed by steps of: forming a graphene layer 2 on the substrate 1; and forming a plurality of nanowires 3 on the graphene layer 2. In addition, the electrode pattern E is formed by steps of: using pulses of the ultrafast laser L to cut and remove the graphene layer 2 and the plurality of nanowires 3 from the substrate 1 along the predetermined path, such that a surface 11 of the substrate 1 is at least partially exposed for forming the electrode pattern E to complete preparation of the gas detection chip 100.

More specifically, the method for producing the gas detection chip includes step S110, step S120, step S130, step S140, and step S150. It should be noted that a sequence of each of the steps described in the present embodiment and the actual operation method can be adjusted according to practical requirements and are not limited to those described in the present embodiment.

As shown in FIG. 1, the step S110 includes providing the substrate 1. The substrate 1 is a flexible polymer substrate. For example, the substrate 1 may be a polyimide (PI) substrate, but the present disclosure is not limited thereto.

In some embodiments of the present disclosure, the substrate 1 may be, for example, a polyethylene (PE) substrate, a polydimethylsiloxane (PDMS) substrate, a polyethylene terephthalate (PET) substrate, a polyethylene naphthalate (PEN) substrate, a polyamide (PA) substrate, a polycarbonate (PC) substrate, or other flexible polymer substrates.

A thickness of the substrate 1 is between 50 micrometers and 300 micrometers, and is preferably between 100 micrometers and 150 micrometers.

Further referring to FIG. 1, the step S120 includes forming the graphene layer 2 on one side surface (e.g., an upper surface) of the substrate 1.

In the present embodiment, the step S120 relates to performing a coating operation, which includes: placing the substrate 1 on a coating machine E1 (e.g., a spin coater), applying a coating solution containing a graphene material on to the surface of the substrate 1 through the coating machine E1 (e.g., a spin coater), and forming the graphene layer 2 by drying a liquid in the coating solution.

Drying conditions for the coating solution may be, for example, to bake the coating solution at a temperature between 130° C. and 150° C. for 1 to 3 hours. Additionally, a thickness of the graphene layer 2 may be, for example, between 10 micrometers and 30 micrometers, and preferably between 15 micrometers and 25 micrometers.

In some embodiments of the present disclosure, the coating solution is formed by dispersing the graphene material in a conductive polymer solution. A conductive polymer of the conductive polymer solution is poly(3,4-ethylenedioxythiophene) (PEDOT), polyphenylene sulfide (PSS), or polyaniline (PANi), but the present disclosure is not limited thereto.

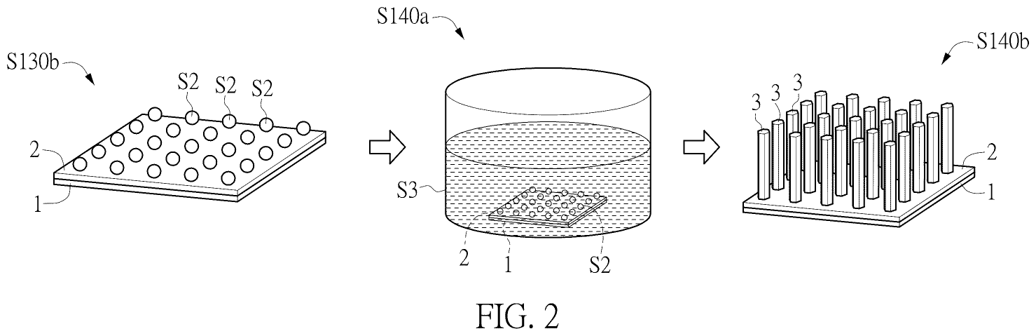

As shown in FIG. 1 and FIG. 2, the step S130 includes: applying (or dropping) a precursor solution S1 on one side surface of the graphene layer 2 (e.g., one side surface away from the substrate 1), and using a heater E2 (e.g., a temperature-controlled heating stage) to heat the precursor solution S1 in order to remove liquid components of the precursor solution S1 (e.g., S130a), thereby forming a plurality of nano seeds S2 (e.g., S130b) on the side surface of the graphene layer 2, and the plurality of nano seeds S2 being dispersed and arranged on the graphene layer 2.

The nano seeds S2 are zinc oxide (ZnO) nano seeds.

In the present embodiment, the precursor solution S1 may include: zinc acetate dihydrate, trimethylamine, and isopropyl alcohol. Accordingly, the precursor solution S1 may be used to form the nano seeds of zinc oxide (ZnO).

The step S140 includes: immersing the substrate 1 with the graphene layer 2 and the plurality of nano seeds S2 formed on the surface in a growth solution S3 (e.g., S140a), so as to form a plurality of nanowires 3 that are dispersed and uprightly oriented on the graphene layer 2 through the plurality of nano seeds S2 (e.g., S140b).

Considering the application of gas detection, a diameter of each of the nanowires 3 can be, for example, between 100 nanometers and 450 nanometers, preferably between 200 nanometers and 400 nanometers, and particularly preferably between 250 nanometers and 350 nanometers. A length of each of the nanowires 3 can be, for example, between 1 micrometer and 10 micrometers, preferably between 1 micrometer and 5 micrometers, and particularly preferably between 1 micrometer and 2 micrometers. In other words, an aspect ratio (length/diameter) of the nanowires 3 can be, for example, between 3 and 10, and preferably between 3 and 7.

In the present embodiment, the thickness of the graphene layer 2 (e.g., between 15 micrometers and 25 micrometers) is greater than the length of each of the nanowires 3 (e.g., between 1 micrometer and 5 micrometers).

More specifically, a distance between any two adjacent nanowires 3 can be, for example, between 100 nanometers and 500 nanometers.

In the present embodiment, the growth solution S3 can include, for example, cyclohexamethylenetetramine, zinc nitrate hexahydrate and water. Furthermore, the growth solution S3 is configured to respectively grow the plurality of nano seeds S2 into the plurality of the nanowires 3 (e.g., zinc oxide nanowires) through a low-temperature hydrothermal method (e.g., at a temperature between 80° C. and 90° C.).

It is worth mentioning that, in the present embodiment, the nanowires 3 are the zinc oxide nanowires as an example, which can be suitable for use as gas detection electrodes, but the present disclosure is not limited thereto. In some embodiments of the present disclosure, the nanowires 3 may also be formed by other functional metal oxides suitable for gas detection, for example, nickel oxide nanowires, tin oxide nanowires, tungsten oxide nanowires, or indium oxide tin nanowires.

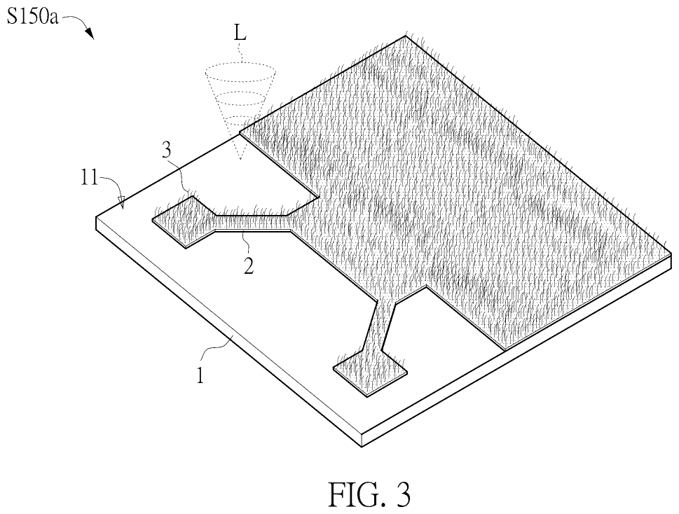

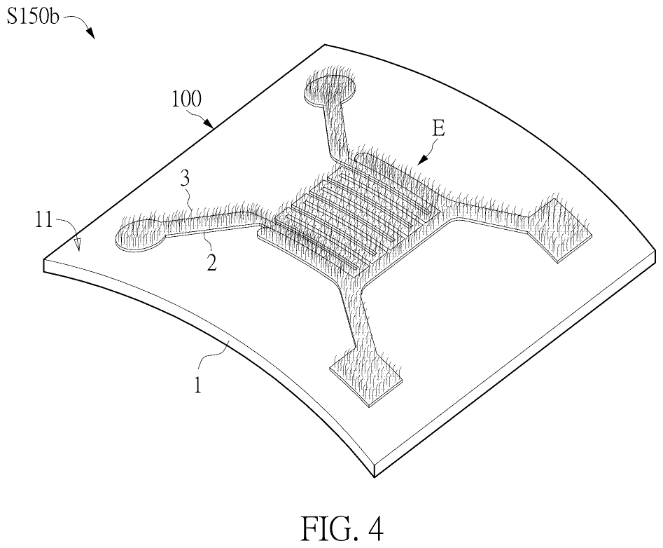

As shown in FIG. 3 and FIG. 4, the step S150 includes: using the pulses of the ultrafast laser L to cut and remove the graphene layer 2 and the plurality of nanowires 3 (e.g., S150a) from the substrate 1 along the predetermined path, such that the surface 11 of the substrate 1 is at least partially exposed for forming the electrode pattern E (e.g., S150b) to complete the preparation of the gas detection chip 100.

More specifically, the ultrafast laser L cuts and removes the graphene layer 2 and the plurality of the nanowires 3, such that the surface 11 of the substrate 1 is at least partially exposed. In addition, the electrode pattern E is composed of the graphene layer 2 and the plurality of nanowires 3 that have not been cut and removed.

The ultrafast laser L refers to a laser light source with a pulse duration in a range of a femtosecond (fs, 10-15 seconds) to a picosecond (ps, 10-12 seconds).

Patterning parameters for using the ultrafast laser L to form the electrode pattern E include: an energy density of between 3 J/cm² and 4 J/cm² (e.g., 3.51 J/cm²), a scanning speed of between 450 mm/s and 550 mm/s (e.g., 500 mm/s), and a repeating frequency of between 800 kHz and 1,200 kHz (e.g., 1000 kHz).

Accordingly, the ultrafast laser L can accurately cut and remove the graphene layer 2 and the plurality of the nanowires 3 to retain the substrate 1 and expose the surface 11 of substrate 1, thereby forming the electrode pattern E composed of the graphene layer 2 (graphene) and the plurality of the nanowires 3 (nanowires).

### First Embodiment

As shown in FIG. 4, an embodiment of the present disclosure provides a gas detection chip 100, which includes a substrate 1 and an electrode pattern E formed on the substrate 1. The electrode pattern E has a micro-nano structure, which is composed of a graphene layer 2 and a plurality of the nanowires 3 (e.g., zinc oxide nanowires) (i.e., ZnO NWs/Gr).

The graphene layer 2 is formed on and in contact with a surface 11 of the substrate 1, and the plurality of the nanowires 3 are dispersed and uprightly oriented on a surface of the graphene layer 2 away from the substrate 1. In addition, the electrode pattern E can expose at least part of the surface 11 of the substrate 1. In other words, the at least part of the surface 11 of the substrate 1, which is not covered by the electrode pattern E, does not have any nanowires 3 distributed on the surface 11.

According to the above configuration, the gas detection chip 100 can be used for nitric oxide (NO) gas detection of 50 ppm to 200 ppm, and can have good detection sensitivity.

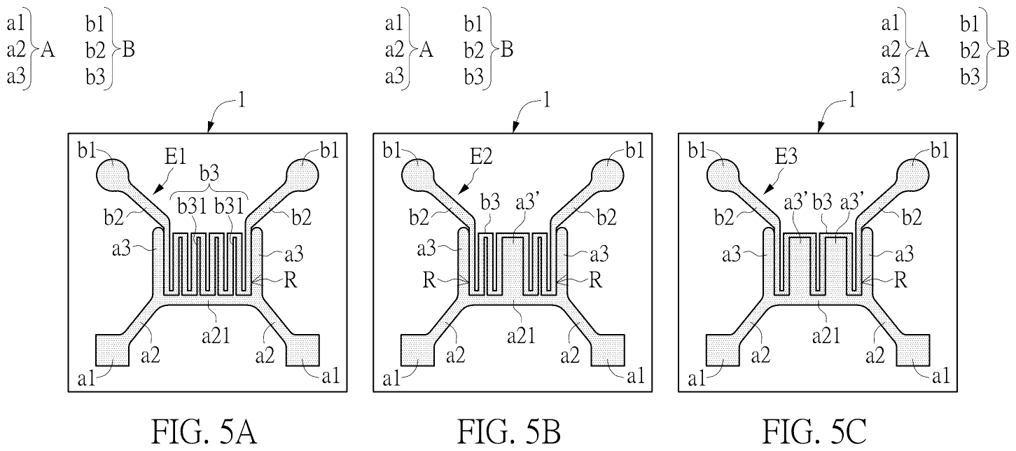

As shown in FIG. 5A, FIG. 5B and FIG. 5C are respectively schematic diagrams of the first to third embodiments of the electrode pattern E according to the present disclosure.

As shown in FIG. 5A, an electrode pattern E1 of the first embodiment includes a first electrode A as a heating electrode and a second electrode B as a sensing electrode. The first electrode A and the second electrode B are disposed in the same layer, and embedded with each other in an interdigital shape without direct contact, but the present disclosure is not limited thereto.

More specifically, the first electrode A has two first contacts a1, two first extension lines a2, an intermediate connecting line a21, and two first interdigitated lines a3. The two first contacts a1 are spaced apart from each other at two of corner positions of the surface 11 of the substrate 1. The two first extension lines a2 are connected to and respectively extend a direction from the two first contacts a1 toward a center of the electrode pattern E1. The intermediate connecting line a21 is connected between two extended ends of the two first extension lines a2. In addition, the two first interdigitated lines a3 respectively extend from two ends of the intermediate connecting line a21 in a direction away from the two first extension lines a2 to form at least one interdigital spacing area R (in the first embodiment, a quantity of the first interdigitated lines a3 of heating electrode is 2).

Furthermore, the second electrode B has two second contacts b1, two second extension lines b2, and a bent extension line pattern b3. The two second contacts b1 are spaced apart from each other at another two of corner positions of the surface 11 of the substrate 1, and are positioned relative to the two first contacts a1. The two second extension lines b2 are connected to and respectively extend a direction from the two second contacts b1 toward the center of the electrode pattern E1, and are close to one end of each of the two first interdigitated lines a3.

The bent extension line pattern b3 has a serpentine shape. In other words, the bent extension line pattern b3 has a plurality of long lines b31 (an interdigital structure of the sensing electrode), which are parallel to and spaced apart from each other, and the plurality of long lines b31 are continuously connected to each other through bends. A quantity of the long lines b31 of the bent extension line pattern b3 (a quantity of interdigital lines of the sensing electrode) is, for example, between 4 and 12 lines, and is preferably between 6 and 10 lines. As shown in FIG. 5A, the quantity of the long lines b31 is 10 lines.

More specifically, the bent extension line pattern b3 is formed in the interdigital spacing area R of the two first interdigitated lines a3, so that the first electrode A and the second electrode B are embedded with each other in the interdigital shape, and a spacing distance between the first electrode A and the second electrode B at their nearest positions (e.g., as the spacing distance between an edge of the bent extension line pattern b3 and an adjacent position of the first interdigitated lines a3) is not less than 20 micrometers. In addition, the interdigital spacing area R is distributed with 4 to 10 lines of the long lines b31 (e.g., 10 lines as in the present embodiment).

### Second Embodiment

As shown in FIG. 5B, a main difference between an electrode pattern E2 of the second embodiment and the electrode pattern E1 of the first embodiment is that the first electrode A further has at least one of a second interdigitated line a3′, which is located inside the two first interdigitated lines a3, the second interdigitated line a3′ extends a direction from a middle section of the intermediate connecting line a21 away from the two first extension lines a2, and is spaced apart from and parallel to the two first interdigitated lines a3. Accordingly, the second interdigitated line a3′ and the two first interdigitated line a3 can jointly divide the interdigital spacing area R into at least two sub-interdigital spacing areas R2 (i.e., a sum of a quantity of the first interdigitated line a3 and the second interdigitated line a3′ of the heating electrode of the second embodiment is 3 lines).

In the present embodiment, a width of the second interdigitated line a3′ is greater than a width of each of the first interdigitated lines a3, and is 1.5 to 3.0 times the width of each of the first interdigitated lines a3.

Furthermore, the bent extension line pattern b3 in the second electrode B is wound around the second interdigitated line a3′, and arranged in the interdigital shape within the two sub-interdigital spacing areas R2. At least two of the plurality of long lines b31 are distributed in each of the sub-interdigital spacing areas R2 (a quantity of the interdigital lines b31 of each of the sub-interdigital spacing areas R2 in the electrode pattern E2 of the second embodiment is 4 lines; that is, the quantity of the interdigital lines b31 of the sensing electrode is 8 lines).

### Third Embodiment

As shown in FIG. 5C, a main difference between an electrode pattern E3 of third embodiment and the electrode pattern E2 of the second embodiment is that a quantity of the second interdigitated lines a3′ of the first electrode A is two, such that the two second interdigitated lines a3′ are arranged to divide the interdigital spacing area R into three sub-interdigital spacing areas R2 (i.e., the sum of the quantity of the first interdigitated line a3 and the second interdigitated line a3′ of the heating electrode of the third embodiment is 4 lines). At least two of the plurality of long lines b31 are distributed in each of the sub-interdigital spacing areas R2 (i.e., the quantity of the interdigital lines b31 of the sensing electrode is 6 lines). According to the above configuration, the design of the electrode patterns E1 to E3 in different embodiments can meet the requirements of detecting different gases in terminal products.

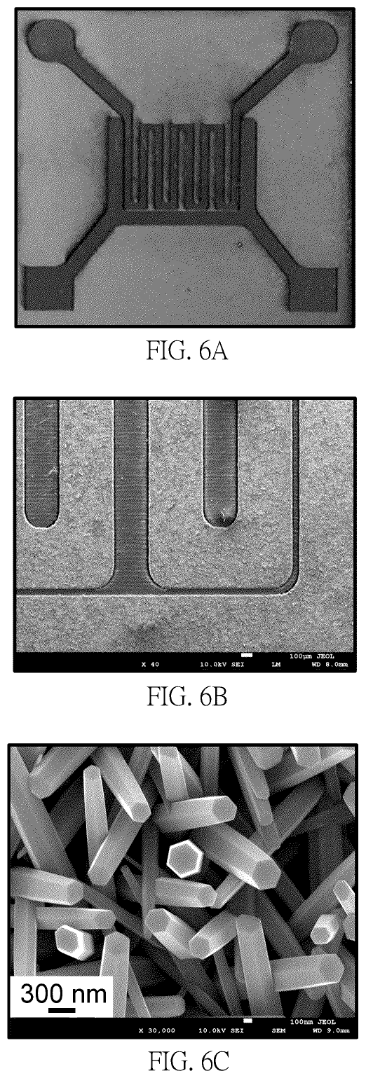

As shown in FIG. 6A is a diagram illustrating the electrode pattern E1 of the first embodiment of FIG. 5A. FIG. 6B is a partially enlarged diagram of the electrode pattern E1 of the first embodiment, which shows a pattern of the bent extension line pattern and the adjacent position of the first interdigitated line. FIG. 6C shows distribution state of the nanowires on the graphene layer according to the embodiment of the present disclosure.

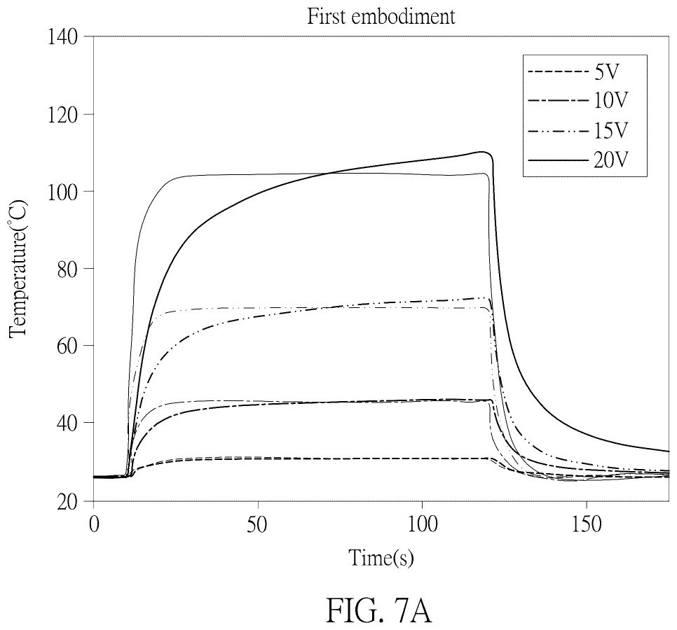

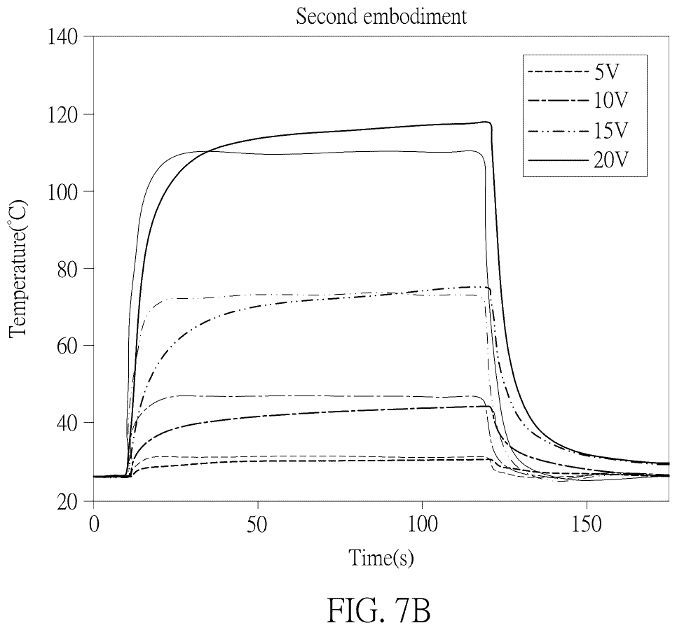

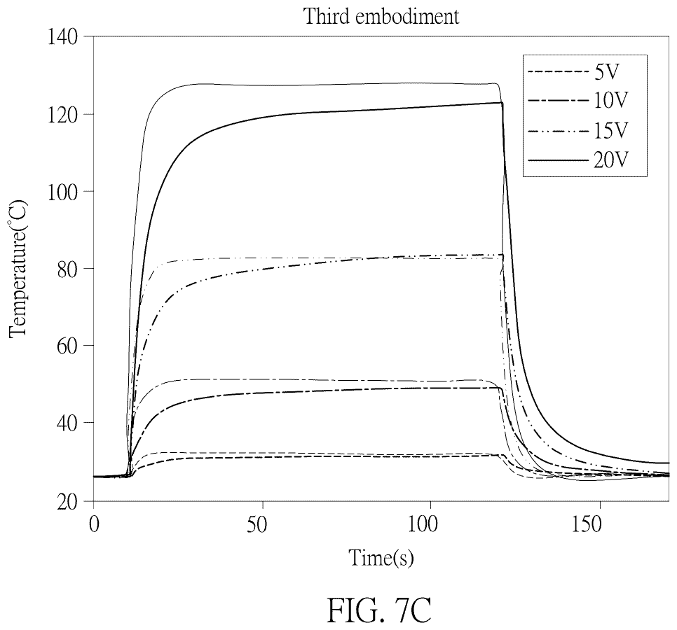

As shown in FIG. 7A, FIG. 7B, and FIG. 7C, FIG. 7A shows the actual and simulated heating response curves of the electrode pattern E1 at different voltages (5V, 10V, 15V, and 20V) according to the first embodiment (in each group of curves, the thick line represents the actual response curve, and the thin line represents the simulated response curve.). FIG. 7B shows the actual and simulated heating response curves of the electrode pattern E2 at different voltages according to the second embodiment. FIG. 7C shows the actual and simulated heating response curves of the electrode pattern E3 at different voltages according to the third embodiment.

As shown in FIG. 7A, FIG. 7B, and FIG. 7C, with the quantity of the interdigitated lines (a sum of the quantity of the first interdigitated lines a3 and/or the second interdigitated lines a3′) of the heating electrode increases, a temperature of the sensing electrode (the second electrode B) rises (especially at higher voltages, such as 20V). This indicates that heat from the heating electrode is effectively distributed to the sensing electrode as the quantity of the interdigitated lines increases, thereby enhancing the sensitivity of the gas detection.

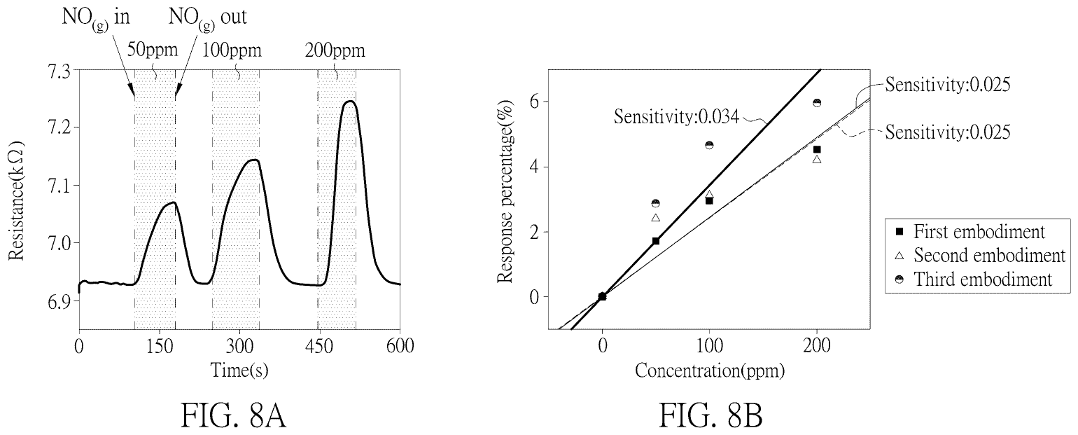

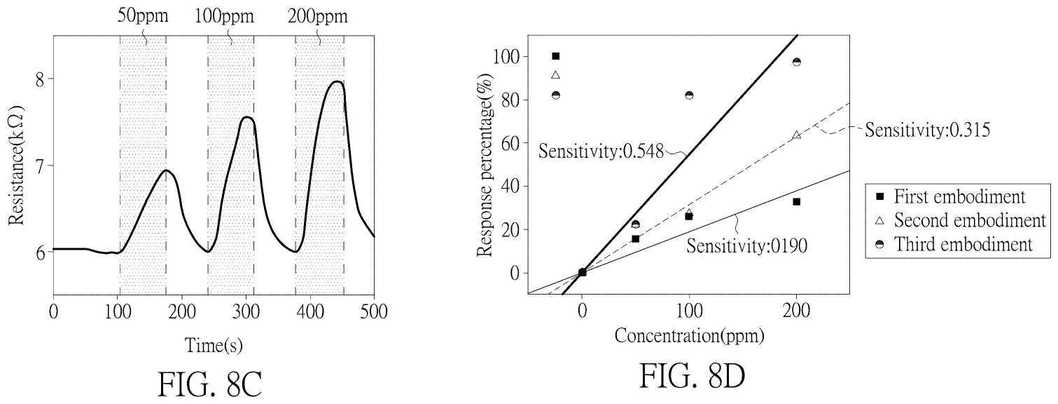

As shown in FIG. 8A, FIG. 8B, FIG. 8C and FIG. 8D show the performance data of the gas detection chip 100 prepared by the electrode pattern E1 to E3 with three different embodiments when detecting nitrogen oxides (NO). The performance data includes time-resolved resistance changes at different concentrations and response percentage and sensitivity at various concentrations of nitrogen oxides.

As shown in FIG. 8A and FIG. 8B, the two sets of data at the top show the NO gas detection results of a sensing element at room temperature (i.e., the sensitivity to NO gas). As shown in FIG. 8C and FIG. 8D, the two sets of data at the bottom show the NO gas detection results of the sensing element under heating (i.e., the sensitivity to NO gas). It can be seen from FIG. 8A, FIG. 8B, FIG. 8C and FIG. 8D that the gas detection chip prepared with the electrode pattern E3 of the third embodiment exhibits the highest gas detection sensitivity (i.e., 0.548) when heated (e.g., when heated to 121° C.).

In summary, the gas detection chip 100, prepared by the electrode pattern E1 to E3 with three different embodiments of the present disclosure, has detection sensitivity for NO gas in the range between 50 ppm and 200 ppm at room temperature, which is approximately between 0.02 and 0.04, and preferably between 0.024 and 0.034.

Furthermore, the gas detection chip 100, prepared by the electrode pattern E1 to E3 with three different embodiments of the present disclosure, has a detection sensitivity for NO gas in the range between 50 ppm and 200 ppm at 120° C. to 125° C., which is approximately between 0.1 and 0.6, and preferably between 0.190 and 0.548.

As shown in FIG. 8A, FIG. 8B, FIG. 8C and FIG. 8D, the response percentage is calculated by the formula: ((NO gas resistance-air gas resistance)/air gas resistance)*100%. The detection sensitivity is obtained by taking the slope of the linear response percentage at different concentrations.

### Beneficial Effects of the Embodiments

Therefore, in the gas detection chip and manufacturing method thereof provided by the present disclosure, by virtue of the above technical solution, the gas detection chip prepared by the above method can be applied to NO gas detection and exhibits high sensitivity.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. A method for producing a gas detection chip, comprising:
providing a substrate;
forming a gas sensing material on the substrate; and
using an ultrafast laser to cut and remove the gas sensing material from the substrate along a predetermined path to form an electrode pattern.

2. The method for producing the gas detection chip according to claim 1, wherein the gas sensing material is selected from the group consisting of graphene and metal oxide, and the gas sensing material has surface topography that includes at least one of: nanowires, nanoparticles, and nanosheets; and wherein the electrode pattern includes at least one of a serpentine shape, a square shape, and a circular shape.

3. The method for producing the gas detection chip according to claim 1, wherein the gas sensing material is formed by steps of:
forming a graphene layer on the substrate; and
forming a plurality of nanowires on the graphene layer;
wherein the electrode pattern is formed by steps of:
using pulses of the ultrafast laser to cut and remove the graphene layer and the plurality of nanowires from the substrate along the predetermined path, such that a surface of the substrate is at least partially exposed for forming the electrode pattern to complete preparation of the gas detection chip.

4. The method for producing the gas detection chip according to claim 3, wherein a thickness of the graphene layer is between 10 micrometers and 30 micrometers, a diameter of each of the nanowires is between 100 nanometers and 450 nanometers, and a length of each of the nanowires is between 1 micrometer and 10 micrometers; and wherein the thickness of the graphene layer is greater than the length of each of the nanowires.

5. The method for producing the gas detection chip according to claim 1, wherein the ultrafast laser is a laser light source with a pulse duration in a range of femtoseconds to picoseconds; and wherein patterning parameters of the ultrafast laser include: an energy density of between 3 J/cm² and 4 J/cm², a scanning speed of between 450 mm/s and 550 mm/s, and a repeating frequency of between 800 kHz and 1,200 kHz.

6. A gas detection chip, comprising:
a substrate; and
an electrode pattern formed on the substrate;
wherein the electrode pattern has a micro-nano structure, and the electrode pattern is formed of a gas sensing material cut by pulses of an ultrafast laser.

7. The gas detection chip according to claim 6, wherein the gas sensing material is composed of a graphene layer and a plurality of nanowires, the graphene layer is formed on and in contact with a surface of the substrate, the plurality of nanowires are dispersed and uprightly oriented on the surface of the graphene layer away from the substrate, and the electrode pattern exposes at least part of the surface of the substrate.

8. The gas detection chip according to claim 6, wherein the electrode pattern includes a first electrode and a second electrode, the first electrode is a heating electrode, the second electrode is a sensing electrode, and the first electrode and the second electrode are disposed in a same layer and embedded with each other.

9. The gas detection chip according to claim 8, wherein the first electrode has:
two first interdigitated lines that defines an interdigital spacing area;
wherein the second electrode has:
a bent extension line pattern having a plurality of long lines, the plurality of long lines being parallel to and spaced apart from each other, and the plurality of long lines being extendedly connected to each other in a bent configuration and formed in the interdigital spacing area; and
wherein the plurality of long lines are defined as interdigitated lines of the sensing electrode, and a quantity of the plurality of long lines is 4 to 12 lines.

10. The gas detection chip according to claim 9, wherein the first electrode further has:
at least one second interdigitated line located inside the two first interdigitated lines to divide the interdigital spacing area into at least two sub-interdigital spacing areas;
wherein the bent extension line pattern of the second electrode is wound around the second interdigitated line, and arranged in an interdigital shape within the two sub-interdigital spacing areas; and wherein at least two of the plurality of long lines are distributed in each of the two sub-interdigital spacing areas.
